(19) United States
(12) Patent Application Publication
(10) Pub. No.: US 2026/0321423 A1
(43) Pub. Date: Oct. 15, 2026

(54) DEEP LEARNING OF DIGITAL PATHOLOGY IMAGES OF PRE-TREATMENT TUMOR BIOPSIES TO PREDICT BREAST CANCER RESPONSE TO CHEMOTHERAPY

(71) Applicants: Ali SADEGHI-NAINI, Maple (CA); Khadijeh SAEDNIA, Toronto (CA); SUNNYBROOK RESEARCH INSTITUTE, Toronto (CA)

(72) Inventors: Ali SADEGHI-NAINI, Maple (CA); Khadijeh SAEDNIA, Toronto (CA); Andrew LAGREE, Toronto (CA); William T. TRAN, Toronto (CA)

(21) Appl. No.: 19/474,023

(22) PCT Filed: Apr. 11, 2024
(86) PCT No.: PCT/CA2024/050459

§ 371 Date: Oct. 9, 2025

Related U.S. Application Data
(60) Provisional application No. 63/495,390, filed on Apr. 11, 2023.

(51) Int. Cl. G16H 50/20; G06T 7/00; G06V 10/25; G06V 10/77; G06V 10/82; G16H 70/60

(52) U.S. Cl. CPC G16H 50/20; G06T 7/0012; G06V 10/25; G06V 10/7715; G06V 10/82; G16H 70/60; G06T 2207/20081; G06T 2207/20084; G06T 2207/30056; G06T 2207/30061; G06T 2207/30068; G06T 2207/30081; G06T 2207/30096; G06V 2201/031

(57) ABSTRACT

A system for automatically predicting chemotherapy response in a patient with cancer using a trained machine learning model, the system including: a digital pathology imaging system for acquiring at least one pathology image; a patch-level processing module for capturing the local correlations within a tumor region in at least one pathology image patch, wherein the at least one pathology image patch is derived from at least one pathology image of a pre-treatment biopsy specimen, and extracting feature maps carrying relevant information of the at least one pathology image patch; a tumor-level processing module for aggregating the local information for each tumor region; a patient-level prediction module for applying the sequence of the local information for the tumor regions to derive global relations and predict the patient's response to chemotherapy.

### FIELD

Aspects of this disclosure relate to deep learning techniques for predicting chemotherapy response in breast cancer patients using digital pathology images of pre-treatment biopsy specimens.

### BACKGROUND

Breast cancer (BC) is the most commonly occurring cancer in women and the second leading cause of cancer-related death in the world.¹ About 20% of breast cancer patients are diagnosed with locally advanced breast cancer (LABC).^{2,3} LABC includes stage III and a subdivision of stage IIB BC, with a tumor size of usually greater than 5 cm that may extend to the skin and/or chest wall, or with axillary lymph node involvement.⁴⁻⁶ LABC patients typically have poorer prognosis compared to early-stage BC due to high risk of cancer progression, local recurrence and metastasis.⁷⁻⁹ LABC is currently treated with neoadjuvant chemotherapy (NAC) followed by surgery and, if required, hormone therapy, targeted therapy, and/or adjuvant radiotherapy.^{10,11} Despite multimodal treatment plans, LABC patients still have low overall survival, highly dependent on tumor response to NAC.¹² Pathological response to NAC has shown high correlations with patient survival.¹³ However, only up to about 30% of LABC patients present pathological complete response (pCR) to standard NAC.^{14,15} The definitive method for pathological assessment of response to NAC is through histopathology on surgical specimens at post-treatment. This limits the opportunity to modify NAC based on tumor response. Therefore, predicting response to NAC either before or early after the treatment initiation could improve therapy outcomes through facilitating personalized patient care. 16,17

Prior research has explored various quantitative imaging approaches for assessing chemotherapy response in breast cancer patients at the diagnosis stage or early after starting the treatment. 17-20 Quantitative biomarkers derived using different medical imaging modalities, including ultrasound, diffuse optical imaging, magnetic resonance imaging (MRI), and positron emission tomography (PET), have shown promise in characterizing breast cancer in terms of responsiveness to chemotherapy particularly when coupled with machine learning (ML) models.²¹⁻²⁴ However, histopathological assessment remains the gold standard approach for cancer diagnosis and characterization in clinical practice. In a recent study, digital histopathological images of diagnostic tumor biopsies have been applied for predicting pCR to NAC in BC patients.²⁵ Several pathomic features were extracted from the segmented nuclei in digital pathology images. A gradient boosting machine (GBM) was adapted for NAC response prediction. The results show the potential of quantitative information derived from the pre-treatment digital pathology images for predicting NAC outcome.

Recent research has explored the efficacy of deep learning (DL) methods in various medical image analysis applications.^{26,27} Previous studies have investigated the performance of various DL frameworks in analyzing histopathological images for tumor grading and subtyping, and predicting the patient survival.²⁸⁻³¹ These studies have demonstrated the potential of the DL models and specifically the deep convolutional networks in digital pathology image analysis. The size of whole slide images (WSIs), however, remains as a constraint in training the DL models, due to memory limitations. Specifically, developing adequate DL models for analyzing the WSIs at high magnification is a challenging task as the size of the high-resolution WSIs can be as large as 150000×150000 pixels.^{32,33} This issue may be addressed by resizing (downscaling) the WSIs and/or extracting small patches from them for analysis by the DL models. Downscaling reduces the image resolution and potentially the efficacy of the information derived by the model. The patching approach requires development of efficient strategies for fusing the patch-level information, while considering the global dependencies, to make relevant conclusions on the WSI level.

Several recent studies have focused on developing efficient DL models for image analysis and classification in computer vision applications. The recent state-of-the-art models are categorized into three general approaches. The first category includes the convolutional neural network (CNN) models. The Xception model³⁴, a well-known model in this group, is an extended version of the Inception-V3 architecture³⁵ in which the Inception modules have been replaced by depth-wise separable convolutions. This model could outperform the Inception-V3 model in classifying the ImageNet dataset.³⁶ The second category comprises the transformers³⁷ that utilize an encoder-decoder architecture with self-attention mechanism.³⁸ The self-attention mechanism in the transformers differentially weights the significance of each part of the input data for the target analysis. The recently introduced vision transformer (ViT) architecture³⁹ has demonstrated a high performance in extracting the global relations in the input images.⁴⁰ However, compared to the CNN-based models, this architecture requires larger amount of training data to yield an adequate generalizability.⁴¹ The last category includes the networks that combine the convolutional layers with the attention mechanisms. The convolutional block attention module (CBAM)⁴², a recently proposed attention mechanism, can be used to infer independent channel and spatial attention maps. The CoAtNet⁴³ is the most recent state-of-the-art architecture that stacks depth-wise convolutional layers and the self-attention mechanism to improve the generalizability of the model.⁴³

### SUMMARY

In one aspect, a system for automatically predicting chemotherapy response in a patient with cancer using a trained machine learning model, the system including:
a digital pathology imaging system for acquiring at least one pathology image;
a patch-level processing module for capturing the local correlations within a tumor region in the at least one pathology image patch, wherein the at least one pathology image patch is derived from at least one pathology image of a pre-treatment biopsy specimen, and extracting feature maps carrying relevant information of the at least one pathology image patch;
a tumor-level processing module for aggregating local information for each tumor region;
a patient-level prediction module for applying a sequence of the local information for the tumor regions to derive global relations and predict the patient's response to chemotherapy.

In another aspect, a computerized method for automatically predicting chemotherapy response in a patient with cancer using a trained machine learning model, the method comprising:
with an imaging system, acquiring at least one image of a pre-treatment biopsy specimen,
identifying at least one region of interest (ROI) comprising a tumor within the at least one image;
applying the at least one image to a trained machine learning model to perform the steps of:
   (a) capturing local correlations within each ROI and extracting feature maps with relevant information of pathology image patches;
   (b) aggregating local information for each ROI; and
   (c) using a sequence of the aggregate information for a plurality of ROIs to derive global relations and predict the patient's response to chemotherapy.

In another aspect, a computerized method for training processing circuitry using machine learning to predict chemotherapy response in a patient with cancer, the method comprising:
training a machine learning model to at least:
capture local correlations within at least one region of interest (ROI) comprising a tumor within at least one image acquired by an imaging system, and extract feature maps with relevant information of pathology image patches derived from the at least one image;
aggregate local information for each ROI; and
and predict the patient's response to chemotherapy by using a sequence of the aggregate local information for a plurality of ROIs to derive global relations.

In another aspect, a non-transitory computer readable medium storing machine-interpretable instruction sets which when executed by a processor, cause the processor to perform a method for predicting chemotherapy response in a patient with cancer, the method comprising:
instantiating a machine learning model architecture;
receiving at least one pathology image patch derived from at least one image of a pre-treatment biopsy specimen acquired by an imaging system;
training the machine learning model architecture using the set of the at least one pathology image patches, the machine learning model architecture including:
   a convolutional network architecture configured to capture local correlations within at least one region of interest (ROI) comprising a tumor within the at least one pathology image patch and extract feature maps with relevant information of pathology image patches;
   a first encoder configured to aggregate local information for each ROI; and
   a second encoder configured to use a sequence of the aggregate information for a plurality of ROIs to derive global relations and predict the patient's response to chemotherapy.

Advantageously, there is provided a hierarchical attention-guided deep learning framework to predict pCR to chemotherapy in breast cancer patients using digital histopathological images of pre-treatment biopsy specimens. The model consists of three modules, including patch-level processing, tumor-level processing, and patient-level response prediction. The hierarchical flow presented in this study could overcome the difficulty of deriving the global relations between different tumor areas in high-resolution WSIs while utilizing the local information within the tumor regions in the analysis.

### BRIEF DESCRIPTION OF THE DRAWINGS

FIG. 1a shows a top-level diagram of an overall system architecture for predicting chemotherapy response in a patient with cancer;

FIG. 1b shows an overview of the preprocessing steps;

FIG. 1c shows a flow diagram depicting exemplary steps for computer-implemented method for predicting chemotherapy response in breast cancer patients using digital histopathological images of pre-treatment biopsy specimens;

FIG. 2a shows stratified random splitting of data at patient level based on the number of patches in each class of the dataset, and includes a box plot with the distribution and quartiles of the number of patches per patient in each class; and FIG. 2b shows the criterion for stratification;

FIGS. 3a-d show a scheme of the hierarchical deep-learning framework for chemotherapy outcome prediction, in which FIG. 3a shows a framework comprising a patch-level sampler for training, and three level of processing, FIG. 3b shows an architecture of a patch-level processing module, FIG. 3c shows an architecture for tumor-level processing module, and FIG. 3d shows an architecture patient-level response prediction module, with the component colors in FIGS. 3b-d corresponding to the associated block in FIG. 3a;

FIG. 4 shows a receiver operating characteristic (ROC) curves on the independent test set for the predictive models developed with two-level (patch-level+patient-level: VIT+ViT, Xception+ViT, and CoAtNet+ViT) and three-level (patch-level+tumor-level+patient-level: ViT+ViT+ViT, Xception+ViT+ViT, and CoAtNet+ViT+ViT) hierarchical framework; and

FIGS. 5a-h show a comparison of Grad-CAM attention heatmaps associated with different patch-level processing modules for two representative tumor regions with a pCR (top row) and non-pCR (bottom row) outcome after NAC, respectively FIGS. 5a-b show tumor areas extracted from the H&E-stained WSIs, FIGS. 5c-d show attention heatmaps of ViT, FIGS. 5e-f show attention heatmaps of Xception+CBAM, FIGS. 5g-h show attention heatmaps of CoAtNet; in which the contours show the tumor bed annotations drawn by a pathologist.

### DETAILED DESCRIPTION

The following detailed description refers to the accompanying drawings. Wherever possible, the same reference numbers are used in the drawings and the following description to refer to the same or similar elements. While embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims.

Moreover, it should be appreciated that the particular implementations shown and described herein are illustrative of the invention and are not intended to otherwise limit the scope of the invention in any way. Indeed, for the sake of brevity, certain sub-components of the individual operating components, and other functional aspects of the systems may not be described in detail herein. Furthermore, the connecting lines shown in the various figures contained herein are intended to represent exemplary functional relationships and/or physical couplings between the various elements. It should be noted that many alternative or additional functional relationships or physical connections may be present in a practical system.

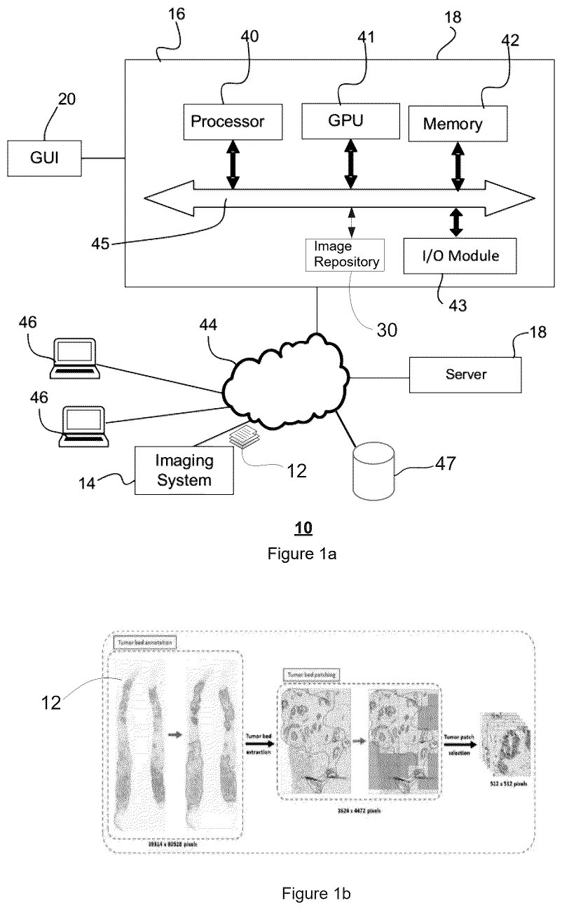

Referring to FIG. 1a, there is shown a top-level diagram of an overall system architecture 10 for predicting cancer response to chemotherapy using digital pathology images 12 of pre-treatment biopsy specimens. The pathology images 12 may be acquired from one or more imaging systems 14. The images 12 captured by the one or more imaging systems 14 are rendered as a digital representation and stored in a computing device 16. The computing device 16 may comprise one or more processing units, such as, graphics processing units (GPUs). In one example, the computing device 16 implements models to predict cancer response to chemotherapy, and output the results via a graphical user interface 20 or a customized plug-in on a peripheral screen.

The computing device 16 comprises an image repository 30 for storage of the images 12. The image repository 30 may be computer readable medium e.g. a hard disk. Alternatively, the acquired images 12 may be stored on a storage server 18 or a computing cloud server. The image repository 30 may also include images of patients for analysis, and/or training images that have been previously analyzed and/or annotated.

The term computing device refers to data processing hardware and encompass all kinds of apparatus, devices, and machines for processing data, including by way of example, a programmable processor, a computer, or multiple processors or computers. The apparatus can also be, or further include, special purpose logic circuitry, e.g., a central processing unit (CPU) 40, a GPU 41 graphics processing unit); a FPGA (field programmable gate array), or an ASIC (application-specific integrated circuit). In some implementations, the data processing apparatus and/or special purpose logic circuitry may be hardware-based and/or software-based. The apparatus can optionally include code that creates an execution environment for computer programs, e.g., code that constitutes processor firmware, a protocol stack, a database management system, an operating system, or a combination of one or more of them. Although a single CPU 40 is illustrated in FIG. 1a, two or more processing units may be used according to particular needs, desires, or particular implementations of the computing device 16. Generally, the GPU 41 executes instructions and manipulates data to perform the operations of the computing device 16. In one example, a GPU 41 is implemented to accelerate computations pertaining to the deep learning methodology.

Memory 42 stores data for the computing device 16 and/or other components of the system 10. Although illustrated as a single memory 42 in FIG. 1a, two or more memories 42 may be used according to particular needs, desires, or particular implementations of the computing device 16. While memory 42 is illustrated as an integral component of the computing device 16 in alternative implementations, memory 42 can be external to the computing device 16 and/or the system. For example, memory 42 comprises computer-readable media (transitory or non-transitory, as appropriate) suitable for storing computer program instructions and data include all forms of non-volatile memory, media and memory devices, including by way of example semiconductor memory devices, e.g., erasable programmable read-only memory (EPROM), electrically erasable programmable read-only memory (EEPROM), and flash memory devices; magnetic disks, e.g., internal hard disks or removable disks; magneto-optical disks; and CD-ROM, DVD+/−R, DVD-RAM, DVD-ROM disks and Blu-ray disks. The memory 42 may store various objects or data, including caches, classes, frameworks, applications, backup data, jobs, web pages, web page templates, database tables, repositories storing business and/or dynamic information, and any other appropriate information including any parameters, variables, algorithms, models, instructions, rules, constraints, or references thereto. Additionally, the memory may include any other appropriate data, such as logs, policies, security or access data, reporting files, as well as others. The processor 40 and the memory 42 can be supplemented by, or incorporated in, special purpose logic circuitry.

In one example, an application in memory 42 comprises algorithmic instructions providing functionality according to particular needs, desires, or particular implementations of the computing device 16, particularly with respect to functionality required for processing modelling calculations for predicting cancer response to chemotherapy.

The computing device 16 may comprise an input/output module 43, to which an input device, such as a keypad, keyboard, touch screen, microphone, speech recognition device, other devices that can accept user information, and/or an output device that conveys information associated with the operation of the computing device 16, including digital data, visual and/or audio information, or a GUI 20.

The computing device 16 comprises an interface, as part of the I/O module 43, used according to particular needs, desires, or particular implementations of the computing device 16. The interface is used by computing device 16 for communicating with other systems in a distributed environment, connected to network 44. Generally, the interface comprises logic encoded in software and/or hardware in a suitable combination and operable to communicate with the network 44. More specifically, the interface may comprise software supporting one or more communication protocols associated with communications. The various components of the computing system are connected by an interconnections means 45, such as an address bus, data bus, a control bus and a peripheral bus.

Client terminals 46 (e.g., remotely located radiology workstations) may request services related to predicting cancer response to chemotherapy and access the results over the communications network 44. Accordingly, the computing device 16 may provide software as a service (SaaS) to the client terminals 46, or provide an application for local download to the client terminals 46, and/or provide functions using a remote access session to the client terminals 46, such as through a web browser. Accordingly, the annotated medical data may be accessible for clinicians on an on-demand basis from the client terminals 46. System 10 may also comprise data storage 47, which is configured to maintain one or more datasets, including data structures storing linkages and other data, such as medical images, libraries, models and rules. Data storage 47 may be a relational database, a flat data storage, a non-relational database, among others.

In one study, the effectiveness of a hierarchical self-attention-guided deep learning framework to predict NAC response in breast cancer patients using digital histopathological images of pre-treatment biopsy specimens was investigated.

### Materials and Methods

The study was conducted in accordance with the institutional ethics review board (IRB) approval from Sunnybrook Health Sciences Centre, Toronto, Canada. The inclusion criteria for the study included: biopsy-confirmed diagnosis of invasive breast cancer, age (18+), and treatment with Anthracycline and/or Taxane based neoadjuvant chemotherapy followed by surgery (any type). Following these criteria, 207 patients were included in the study. The patients aged between 28-79 years with a mean and standard deviation of 51.1+10.4 years. The patients had an initial tumor size in the range of 1.1-15.5 with a mean and standard deviation of 5.01+2.9. The clinical N stage was 0 (no positive lymph nodes) for 24.6% (=51); 1 (1-3 positive lymph nodes) for 66.2% (=137); 2 (4-9 positive lymph nodes) for 8.2% (=17); and 3 (>10 positive lymph nodes) for 1% (=2) of the patients. Among 207 patients, 62.3%, 54.1%, 62.3% and 41.5% had tumors with an ER+, PR+, and HER2+ receptor status, respectively. Most of the patients (=192; 93%) had invasive ductal carcinoma (IDC), with a minority (=15; 7%) diagnosed with invasive lobular carcinoma (ILC).

All patients had a breast core needle biopsy before NAC with a pathological review as part of the standard of care. The formalin-fixed paraffin embedded (FFPE) blocks containing core biopsy specimens were microtomed into 4 μm sections. The specimen sections were prepared onto histopathology slides and stained with hematoxylin and eosin (H&E). Digital histopathological images of the H&E-stained slides were acquired using a digital pathology imaging system (Huron Digital Pathology, St. Jacob's Canada). The images were acquired at 40× (pixel size: 0.2 μm) for 125 all patients. The digital WSIs were manually reviewed for artifacts or occlusions within the specimen before analysis and any slides associated with a distorted or blurry image was re-imaged.

The treatment response was assessed for each patient after surgery and categorized into pathological complete response (pCR) versus pathological non-complete response (non-pCR), as ground truth labels for evaluating the developed models. A standard assessment method using the residual cancer burden index (RCBI) was applied for assessment of treatment response. An RCBI score of 0 (i.e., pCR) was defined as the absence of residual invasive and nodal disease.⁴⁴ Patients who demonstrated residual disease were classified as non-pCR (i.e., RCBI >0).⁴⁴ All pathology reviews (pre-treatment and post-surgery histopathology) were performed by board-certified breast pathologists as part of the patient's standard of care. The pathological evaluations after surgery demonstrated 25.2% (=52) of the patients with a pCR, and 74.8% (=155) with a non-pCR.

### Preprocessing and Dataset Splitting

FIG. 1c shows a flow diagram 200 outlining exemplary steps of a computer-implemented method for predicting NAC response in breast cancer patients using digital histopathological images of pre-treatment biopsy specimens in accordance with various implementations described herein. It should be understood that while the operational flow diagram indicates a particular order of execution of the operations, in other implementations, the operations might be executed in a different order. Further, in some implementations, additional operations or blocks may be added to the method. Likewise, some operations or blocks may be omitted.

In step 202, digital histopathological images of the H&E-stained slides were acquired using a digital pathology imaging system (Huron Digital Pathology, St. Jacob's Canada). The images were acquired at 40× (pixel size: 0.2 μm). The digital WSIs were manually reviewed for artifacts or occlusions within the specimen before analysis and any slides associated with a distorted or blurry image were re-imaged.

In step 204, the tumor bed areas on the WSIs were manually annotated. In one example, a slide digital pathology image management and workflow was used for annotations, such as a Sedeen software package⁴⁵, from PathCore Inc., Toronto, Canada. The tumor bed annotations were pre-processed on the three-channel RGB images for patch extraction. Once the data was preprocessed, patches or portions or “chunks” of data are created with a certain size and format prior to being fed into a trained network for processing. Based on learned patterns, nodes, and connections, the trained network determines outputs based on the input patches, as will be described in detail below.

Tumor margins were included, when required, to obtain non-overlapping patches with a size of 512×512 pixels (pixel size: 0.2 μm) from the annotated tumor regions. However, only the patches with more than 10% tumor tissue and less than 10% white background were retained for the study (FIG. 1b). In total, 1,733,421 patches were included in this study.

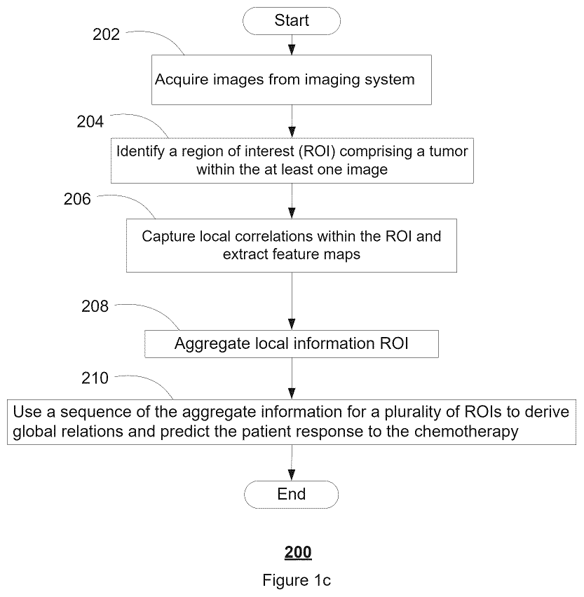

Out of the 207 patients in this study, 155 and 52 patients were labeled as the non-pCR and pCR, respectively. The number of patches for each patient varied from 30 to 14418 (median=1755). The non-pCR and pCR classes included 1,423,210 and 310,211 patches, respectively. As such, the ratio of the pCR to non-pCR class at the patient- and patch-level was 25.2% and 17.9%, respectively. FIG. 2a shows the distribution and quartiles of the number of patches per patient in the pCR and non-pCR classes of the dataset. A stratified random splitting approach was applied to split the data at patient level into the training (70%; n=144 with 9,430 annotated tumor beds and 1,559,784 patches) and test sets (30%; n=63 with 3,574 annotated tumor beds and 173,637 patches). The stratified random splitting was performed considering the response label and the number of extracted patches for each patient. The first quartile, median and the third quartile of the number of patches in each response class (pCR and non-pCR) were used to stratify the patients into eight different bins for random sampling, as shown in FIG. 2b. A similar procedure was used to utilize a five-fold cross-validation on the training set for optimizing the framework's hyperparameters (described further in System Training section below). The training and test sets obtained using the stratified random splitting approach were assessed, using statistical tests, for possible inhomogeneities in terms of clinical feature distribution between the two sets. The Pearson's chi-squared homogeneity test was used for categorical variables. The continuous variables were assessed using the t-test. Table 1 presents the clinical information of the patients in the training and test sets, along with the statistical test results.

TABLE 1
Demographic and clinical characteristics of the patients. The
distribution of each variable was compared between the
training and test sets using the Pearson's chi-squared homogeneity
test for categorical variables and the t-test for continuous
variables; the p-values are reported in the last column.

| Patient demo- | | | | | |
|---|---|---|---|---|---|
| graphics and | Count (%) | | | | |
| clinico- | Training (n = 144) | | Test (n = 63) | | p-value |
| pathological | PCR | non-pCR | PCR | non-pCR | p = |
| characteristics | (n = 36) | (n = 108) | (n = 16) | (n = 47) | 0.91 |
| Median Age | 47.5 | 51 | 49.5 | 51 | p = |
| (Years) | | | | | 0.61 |
| Menopausal | | | | | |
| Status | | | | | |
| Pre/Peri- | 15 (42%) | 56 (52%) | 9 (56%) | 24 (51%) | p = |
| menopausal | | | | | 0.69 |
| Post- | 21 (58%) | 52 (48%) | 7 (44%) | 23 (49%) | |
| menopausal | | | | | |
| Tumor Size | | | | | |
| Mean | 39.8 ± | 55.8 ± | 37.1 ± | 49.6 ± | p= |
| Tumor Size | 21.1 | 29.5 | 23.9 | 30.7 | 0.22 |
| (mm; ±SD) | | | | | |
| Nodal Status | | | | | |
| (N Stage) | | | | | |
| No Positive | 14 (39%) | 22 (21%) | 7 (44%) | 8 (17%) | p = |
| Lymph Nodes | | | | | 0.59 |
| (N0) | | | | | |
| 1-3 Positive | 21 (58%) | 75 (69%) | 8 (50%) | 33 (70%) | |
| Lymph Nodes | | | | | |
| (N1) | | | | | |
| 4-9 Positive | 1 (3%) | 10 (9%) | 1 (6%) | 5 (11%) | |
| Lymph Nodes | | | | | |
| (N2) | | | | | |
| ≥10 Positive | 0 (0%) | 1 (1%) | 0 (0%) | 1 (2%) | |
| Lymph Nodes | | | | | |
| (N3) | | | | | |
| Receptor Status | | | | | |
| ER Positive | 13 (36%) | 79 (73%) | 3 (19%) | 34 (72%) | p = |
| | | | | | 0.81 |
| PR Positive | 12 (33%) | 68 (63%) | 2 (13%) | 30 (64%) | p = |
| | | | | | 0.27 |
| HER2 Positive | 24 (67%) | 38 (35%) | 10 (63%) | 14 (30%) | p = |
| | | | | | 0.51 |
| Histology | | | | | |
| Invasive | 36 (100%) | 96 (89%) | 16 (100%) | 44 (94%) | p = |
| Ductal | | | | | |
| Carcinoma | | | | | 0.53 |
| Invasive | 0(0%) | 12 (11%) | 0 (0%) | 3 (6%) | |
| Lobular | | | | | |
| Carcinoma | | | | | |
| Nottingham | | | | | |
| Grade | | | | | |
| 1 | 1 (3%) | 4 (4%) | 0 (0%) | 1 (2%) | p = |
| 2 | 9 (25%) | 39 (36%) | 2 (13%) | 22 (47%) | 0.47 |
| 3 | 26 (72%) | 65 (60%) | 14 (87%) | 24 (51%) | |
| Other Clinical | | | | | |
| Information | | | | | |
| Inflammatory | 2 (6%) | 11 (10%) | 2 (13%) | 5 (11%) | p = |
| Breast Cancer | | | | | 0.64 |

### Response Prediction Framework

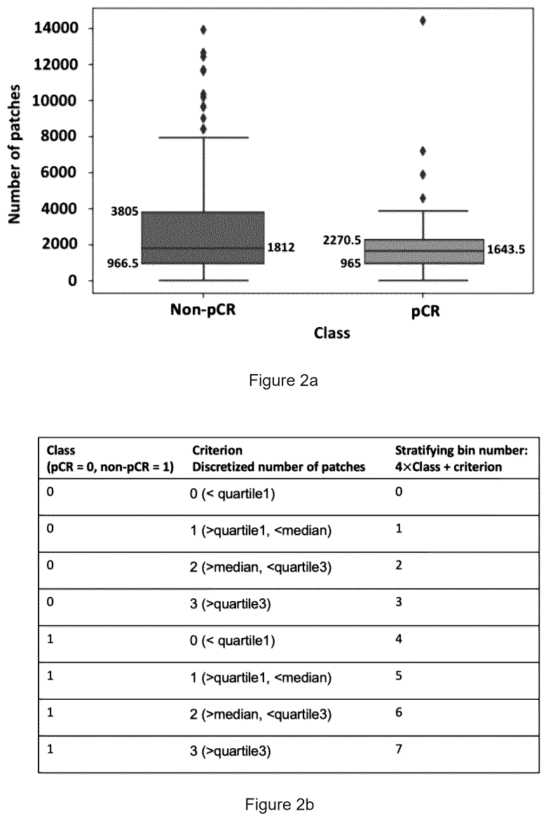

FIGS. 3a-c show a scheme of an exemplary hierarchical deep-learning framework 300 for therapy response prediction. The framework 300 includes a patient-wise sampler 302 which addresses the unbalance issue of the training data at patient and patch levels. The sampler 302 applies a weighted sampling strategy for under-sampling the majority class in the training set based on the total number of patches and the number of patients in each class:

$${w}_{p} = \frac{{S}_{pt}}{{S}_{pp} \times {S}_{pc}}$$

where wp is the calculated weight for each patient, Sₚₜ is the total number of patches in the training set, Sₚₚ is the total number of patches for the patient, and S_{pc} is the total number of patches in the associated class of the patient (pCR or non-pCR). After calculating the weights for all patients in the training set, they are normalized such that the sum of all weights adds up to one. All patches of each patient are assigned equal weights for selection by the sampler. The sampler size (number of patches in each epoch) is tuned as a hyperparameter during the system's training process.

The overall framework 300 includes a hierarchical flow of the patch-level processing module 304, tumor-level processing module 306, and the patient-level response prediction module 308. The framework 300 uses a self-attention-guided convolutional network architecture 310 followed by two customized ViT network architectures 312, 314 for a hierarchical deep-learning-based analysis of the digital pathology images for NAC response prediction.

In the patch-level processing module 302, a modified CoAtNet model with two convolutional components followed by two self-attention modules (FIG. 3b) extracts the descriptive features from the pathology image patches.

Step 206: In one example, the input patches are downsampled to 256×256 pixels using a two-layer convolutional block. For example, a feature map of size 768 associated with each patch is collected from the last layer (global pooling layer) before the fully connected layer in the network. The sequence of the feature maps for each tumor bed is generated by stacking the sorted patch-level feature maps of the corresponding tumor bed annotation in the associated digital pathology image. The feature maps may be sorted based on the position of their associated patch in the tumor bed from top left to bottom right.

Step 208: The generated feature map sequence is fed as input to the first ViT model 312 that includes sixteen encoder blocks to explore the relations in aggregated features of each tumor bed. The initial positional encoding vector for the tumor-level processing module 306 is defined based on the generated sequence of the sorted patches.

To adapt the sequence of feature maps for the ViT architecture, the input size is defined as the (sequence length×size of feature map vector). Since the number of patches varies for different tumor beds, a threshold is set for the maximum length of the sequence (tuned as a hyperparameter during the system's training process). For the tumor beds with a smaller number of patches than the threshold, a zero-masking approach is applied, while for the beds with a larger number of patches, the starting patch of sequence is randomly selected.

Step 210. A similar approach as described in paragraphs and is applied in the patient-level response prediction network 314 to aggregate the information obtained at the tumor-level processing and exploiting the global dependencies between the tumor areas for therapy response prediction. A feature map of size 1024 associated with each tumor bed is extracted from the last layer (mean pooling layer) of the tumor-level processing ViT before the fully connected layer. A sequence of the feature maps is generated for each patient to feed into the patient-level response prediction ViT network 314 that includes two encoder blocks. Similar feature map sorting and sequence length thresholding strategies applied for the tumor-level processing ViT 312 are used to create the input sequence of this ViT network 314.

### System Training

The framework 300's hyperparameters were optimized using a grid search approach with five-fold cross validation on the training set. The sampler size was tuned to 10000 patches for each training epoch. The number of patches per tumor bed, and the number of tumor bed regions per patient varied between 1 to 3879 (median=6), and 1 to 285 (median=44), respectively. Accordingly, the threshold for maximum length of the sequence in the tumor-level and patient-level processing modules was tuned to 10 and 50, respectively. These thresholds resulted in an input size of 10×768 and 50×1024 for the associated networks, respectively. The batch size was tuned to 200 for all three networks. A maximum number of 100 epochs was used for training each network. The learning rate was tuned to 0.05 for the patch-level and tumor-level processing networks and 0.01 for the patient-level response prediction network. After the hyperparameter tuning, 20% of the training set patients (n=29) were selected as the validation set using the stratified random splitting approach and the framework with optimized hyperparameters was trained using the remaining patients in the training set (80% of the training set, n=115). Early stopping was utilized for all networks based on the validation loss during the training process to prevent overfitting.

### Evaluation

The performance of the framework was assessed on the independent unseen test set using the accuracy, sensitivity, specificity, loss, F1-score, and area under the receiver operating characteristics (ROC) curve (AUC). A threshold value of 0.5 was used as the cut-off to calculate the sensitivity and specificity. To evaluate the performance and effectiveness of the hierarchical framework, ablation experiments were performed with models that only incorporated the patch-level processing, patch-level+tumor-level, and patch-level+patient-level processing modules. In another set of experiments, maximum voting was applied on the output of the patch-level and patch-level+tumor-level models to obtain the tumor-level and patient-level response prediction results, for comparison with those of the hierarchical models. Specifically, the maximum voting was applied over the responses (pCR/non-pCR) predicted by the patch-level or patch-level+tumor-level model for all the patches/tumor regions associated with a tumor region/patient. Separate sets of experiments were conducted using two other frameworks that utilized an Xception model coupled with CBAM attention as a state-of-the-art CNN-based model, and a pure self-attended architecture with a ViT model as their patch-level processing network. A gradient class activation map (Grad-CAM) approach⁴⁶ was applied for visualization of the trained patch-level processing attention mechanisms. The Grad-CAM approach provides information on salient regions in an image for a specific class to permit interpreting the network decisions based on the model attention. The attention heatmaps were generated for the patches presented to the trained framework based on their predicted label. The generated heatmaps were stitched together using their position information to create the complete attention maps for individual tumor areas. The visualization heatmaps were reviewed to assess and compare the efficacy of attention mechanisms in different networks.

### Results

Table 2 shows the performance of the framework compared with other similar models.

The results demonstrate that the CoAtNet architecture as the patch-level processing module outperformed the Xception model with CBAM attention and the ViT model, with an accuracy of 81% on the test set, compared to accuracies of 79% and 78% respectively. Results of the ablation experiments demonstrate that the three-level hierarchical frameworks could outperform the patch-level only and the two-level (patch-level+tumor-level and patch-level+patient-level) processing frameworks. The patch-level+tumor-level and patch-level+patient-level processing frameworks consisting of cascaded ViT models resulted in an AUC of 0.78 and 0.77, respectively, on the test set, while the similar architecture with three-level hierarchy resulted in an AUC of 0.80. Also, the two-level hierarchical models (patch-level+tumor-level and patch-level+patient-level) with the Xception+CBAM architecture followed by the ViT module could achieve an AUC of 0.80 and 0.82, respectively, whereas the similar model with three levels of processing could achieve an AUC of 0.86. The tumor-level and patient-level results obtained through maximum voting on the outputs of the patch-level and patch-level+tumor-level models demonstrate a better performance of the corresponding hierarchical models in response prediction. The best performance was achieved by the framework with three-level hierarchy consisting of the CoAtNet architecture as a patch-level processing module and two ViT architectures for the tumor-level processing and patient-level response prediction. This model resulted in an accuracy of 86% on the test set, and a sensitivity, specificity, F1-score and AUC of 87%, 83%, 90% and 0.89 respectively.

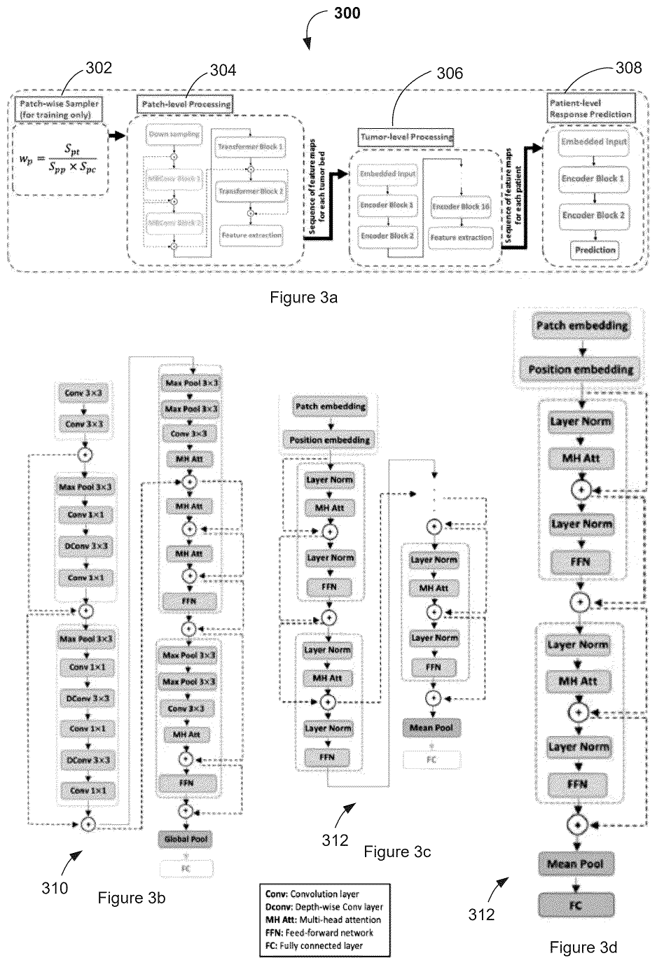

FIG. 4 compares the AUC of the two-level and three-level hierarchical architectures with different patch-level processing modules (ViT, Xception+CBAM, and CoAtNet). The AUCs are in the range of 0.77 and 0.89 with the best results associated with the three-level hierarchical framework using the CoAtNet as the patch-level processing component.

TABLE 2
Performance of different architectures in predicting NAC response using pre-treatment
digitized pathology slides of core needle biopsies. The best value in each column is in bold.

| | | | | | | | | | | Independent Test set | | | | | |
|---|---|---|---|---|---|---|---|---|---|---|---|---|---|---|---|
| | | Training Set | | | | Validation Set | | | | | | | | | F1- |
| Model | | Loss | Ace | Sens | Spec | Loss | Acc | Sens | Spec | Loss | Acc | Sens | Spec | AUC | score |
| VIT + | Patch-level | 0.61 | 0.76 | 0.76 | 0.75 | 0.61 | 0.77 | 0.78 | 0.74 | 0.59 | 0.78 | 0.79 | 0.75 | 0.78 | 0.84 |
| (None/ | Max-voting on | — | 0.73 | 0.73 | 0.73 | — | 0.74 | 0.75 | 0.72 | — | 0.74 | 0.75 | 0.73 | — | 0.82 |
| VIT) + | Patch-level | | | | | | | | | | | | | | |
| | (Tumor-level | | | | | | | | | | | | | | |
| | Results) | | | | | | | | | | | | | | |
| (None/ | Max-voting on | — | 0.72 | 0.73 | 0.72 | — | 0.72 | 0.73 | 0.71 | — | 0.73 | 0.74 | 0.72 | — | 0.81 |
| VIT) | Patch-level | | | | | | | | | | | | | | |
| | (Patient-level | | | | | | | | | | | | | | |
| | Results) | | | | | | | | | | | | | | |
| | Patch-level + | 0.60 | 0.77 | 0.78 | 0.75 | 0.60 | 0.78 | 0.79 | 0.75 | 0.59 | 0.79 | 0.80 | 0.75 | 0.78 | 0.85 |
| | Tumor-level | | | | | | | | | | | | | | |
| | Max-voting on | — | 0.76 | 0.77 | 0.75 | — | 0.76 | 0.77 | 0.74 | — | 0.78 | 0.79 | 0.75 | — | 0.84 |
| | Patch-level + | | | | | | | | | | | | | | |
| | Tumor-level | | | | | | | | | | | | | | |
| | (Patient- | | | | | | | | | | | | | | |
| | level Results) | | | | | | | | | | | | | | |
| | Patch-level + | 0.59 | 0.79 | 0.79 | 0.78 | 0.60 | 0.78 | 0.79 | 0.76 | 0.57 | 0.80 | 0.81 | 0.78 | 0.77 | 0.86 |
| | Patient-level | | | | | | | | | | | | | | |
| | Patch-level + | 0.57 | 0.80 | 0.80 | 0.79 | 0.58 | 0.79 | 0.80 | 0.76 | 0.55 | 0.82 | 0.83 | 0.79 | 0.80 | 0.87 |
| | Tumor-level + | | | | | | | | | | | | | | |
| | Patient-level | | | | | | | | | | | | | | |
| | Patch-level | 0.57 | 0.77 | 0.80 | 0.74 | 0.58 | 0.77 | 0.79 | 0.73 | 0.56 | 0.79 | 0.80 | 0.76 | 0.79 | 0.85 |
| | Max-voting on | — | 0.76 | 0.78 | 0.75 | — | 0.77 | 0.78 | 0.74 | — | 0.78 | 0.79 | 0.76 | — | 0.84 |
| | Patch-level | | | | | | | | | | | | | | |
| | (Tumor-level | | | | | | | | | | | | | | |
| | Results) | | | | | | | | | | | | | | |
| Xception | Max-voting on | — | 0.76 | 0.77 | 0.76 | — | 0.76 | 0.77 | 0.74 | — | 0.76 | 0.77 | 0.75 | — | 0.83 |
| (+CBAM) + | Patch-level | | | | | | | | | | | | | | |
| (None/ | (Patient-level | | | | | | | | | | | | | | |
| VIT) + | Results) | | | | | | | | | | | | | | |
| (None/ | Patch-level + | 0.55 | 0.80 | 0.81 | 0.77 | 0.56 | 0.80 | 0.81 | 0.75 | 0.55 | 0.81 | 0.82 | 0.77 | 0.80 | 0.86 |
| ViT) | Tumor-level | | | | | | | | | | | | | | |
| | Max-voting on | — | 0.78 | 0.79 | 0.77 | — | 0.79 | 0.80 | 0.75 | — | 0.79 | 0.80 | 0.76 | — | 0.85 |
| | Patch-level + | | | | | | | | | | | | | | |
| | Tumor-level | | | | | | | | | | | | | | |
| | (Patient- | | | | | | | | | | | | | | |
| | level Results) | | | | | | | | | | | | | | |
| | Patch-level + | 0.54 | 0.82 | 0.83 | 0.79 | 0.53 | 0.82 | 0.84 | 0.79 | 0.53 | 0.83 | 0.84 | 0.81 | 0.82 | 0.88 |
| | Patient-level | | | | | | | | | | | | | | |
| | Patch-level + | 0.52 | 0.84 | 0.84 | 0.84 | 0.51 | 0.84 | 0.85 | 0.82 | 0.50 | 0.85 | 0.86 | 0.83 | 0.86 | 0.90 |
| | Tumor-level + | | | | | | | | | | | | | | |
| | Patient-level | | | | | | | | | | | | | | |
| CoAtNet + | Patch-level | 0.56 | 0.78 | 0.80 | 0.76 | 0.58 | 0.78 | 0.80 | 0.74 | 0.54 | 0.81 | 0.82 | 0.78 | 0.79 | 0.86 |
| (None/ | Max-voting on | — | 0.76 | 0.77 | 0.76 | — | 0.77 | 0.78 | 0.74 | — | 0.78 | 0.80 | 0.77 | — | 0.85 |
| VIT) + | Patch-level | | | | | | | | | | | | | | |
| (None/ | (Tumor-level | | | | | | | | | | | | | | |
| ViT) | Results) | | | | | | | | | | | | | | |
| | Max-voting on | — | 0.76 | 0.77 | 0.75 | — | 0.76 | 0.77 | 0.74 | — | 0.77 | 0.79 | 0.76 | — | 0.84 |
| | Patch-level | | | | | | | | | | | | | | |
| | (Patient-level | | | | | | | | | | | | | | |
| | Results) | | | | | | | | | | | | | | |
| | Patch-level + | 0.54 | 0.80 | 0.83 | 0.78 | 0.55 | 0.80 | 0.81 | 0.76 | 0.53 | 0.82 | 0.83 | 0.78 | 0.81 | 0.87 |
| | Tumor-level | | | | | | | | | | | | | | |
| | Max-voting on | — | 0.78 | 0.80 | 0.75 | — | 0.79 | 0.80 | 0.76 | — | 0.80 | 0.81 | 0.78 | — | 0.86 |
| | Patch-level + | | | | | | | | | | | | | | |
| | Tumor-level | | | | | | | | | | | | | | |
| | (Patient- | | | | | | | | | | | | | | |
| | level Results) | | | | | | | | | | | | | | |
| | Patch-level + | 0.52 | 0.83 | 0.85 | 0.80 | 0.56 | 0.81 | 0.83 | 0.78 | 0.51\| | 0.85 | 0.86 | 0.80 | 0.84 | 0.89 |
| | Patient-level | | | | | | | | | | | | | | |
| | Patch-level + | 0.48 | 0.85 | 0.86 | 0.84 | 0.52 | 0.84 | 0.85 | 0.81 | 0.48 | 0.86 | 0.87 | 0.83 | 0.89 | 0.90 |
| | Tumor-level + | | | | | | | | | | | | | | |
| | Patient-level | | | | | | | | | | | | | | |

Acc: accuracy,
Sens: sensitivity,
Spec: specificity,
AUC: area under the ROC curve.

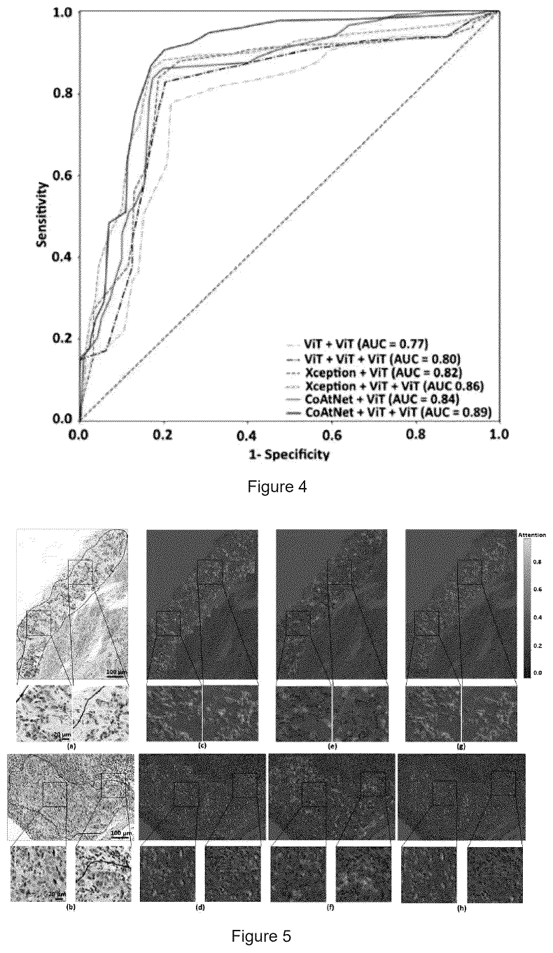

FIG. 5 shows attention heatmaps obtained for two representative tumor regions: one with a pCR and the other one with a non-pCR outcome after NAC. The heatmaps were generated for each patch in the tumor bed region (contoured by an expert pathologist) using the Grad-CAM approach and overlaid on the original pathology image for visualization. A comparison between the heatmaps associated with different patch-level processing modules shows that the CoAtNet architecture has demonstrated more focus on the tumor regions compared to the other two networks.

Accordingly, in this example the hierarchical deep learning framework 300 is capable of predicting breast cancer response to NAC using digital histopathological images of pre-treatment biopsy specimens of the patients in an independent test set with a sensitivity, specificity, and F1-score of 87%, 83%, and 90%, respectively. A comparison between the attention heatmaps generated by various patch-level processing architecture demonstrates that the CoAtNet architecture paid more attention to the tumor areas compared to surrounding the healthy tissues. The performance of the hierarchical model shows that the combination of convolutional blocks with self-attention modules adapted as the patch-level processing component can effectively extract local information within the tumor patches, while the relations between these features in each tumor area and at patient-level for NAC response prediction can be successfully modeled using the vision transformer modules.

The hierarchical framework 300 thus provides an effective approach for analyzing the WSIs at high-resolution. Results of the ablation experiments in this study demonstrate that the three-level hierarchical model could outperform the patch-level only and the two-level hierarchical models in predicting the NAC response. The performance of a purely convolutional architecture, a completely self-attention-based model, and a combination of convolutional and self attention components in extracting informative features from the tumor patches were compared. The results demonstrate that coupling the convolutional and self-attention modules is a more effective architecture in extracting local features. The positional embedding approach in this study enables the framework to extract global relations within and between different tumor areas and predict the pCR/non-pCR outcome for each patient using multi-head attention modules.

In another implementation, automated tumor annotation methods may be employed to annotate tumor beds.

In another implementation, the framework analyses the pathology image patches with a pre-processing step for nucleus detection and tumor cell classification, thereby extract tumor microenvironment features of the cancer cells specifically.

In another implementation, the methods described herein may be adapted for the analysis of digital pathology WSIs in other applications. This includes diagnostic and prognostic applications for different cancer types such as the breast51, prostate52, liver53, and the lung cancer54.

Each of the non-limiting aspects or examples described herein can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact discs and digital video discs), magnetic cassettes, memory cards or sticks, random access memories (RAMs), read only memories (ROMs), and the like.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can he in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

### REFERENCES

1. Siegel R L, Miller K D, Fuchs H E, Jemal A. Cancer statistics, 2022. C A Cancer J Clin. 2022; 72(1):7-33. doi:10.3322/caac.21708
2. Tryfonidis K, Senkus E, Cardoso M J, Cardoso F. Management of locally advanced breast cancer-perspectives and future directions. Nat Rev Clin Oncol. 2015; 12(3):147-162. doi:10.1038/nrclinonc.2015.13
3. Giordano S H. Update on Locally Advanced Breast Cancer. Oncologist. 2003; 8(6):521-530. doi:10.1634/theoncologist.8-6-521
4. Dhanushkodi M, Sridevi V, Shanta V, et al. Locally Advanced Breast Cancer (LABC): Real-World Outcome of Patients From Cancer Institute, Chennai. JCO Glob Oncol. 2021; (7):767-781. doi:10.1200/G O.21.00001
5. Sadeghi-Naini A, Papanicolau N, Falou O, et al. Quantitative Ultrasound Evaluation of Tumor Cell Death Response in Locally Advanced Breast Cancer Patients Receiving Chemotherapy. Clinical Cancer Research. 2013; 19(8):2163-2174. doi:10.1158/1078-0432.CCR-12-2965
6. Moghadas-Dastjerdi H, Sha-E-Tallat H R, Sannachi L, Sadeghi-Naini A, Czarnota G J. A priori prediction of tumour response to neoadjuvant chemotherapy in breast cancer patients using quantitative C T and machine learning. Sci Rep. 2020; 10(1):10936. doi:10.1038/s41598-020-67823-8
7. Falou O, Sadeghi-Naini A, Prematilake S, et al. Evaluation of Neoadjuvant Chemotherapy Response in Women with Locally Advanced Breast Cancer Using Ultrasound Elastography. Transl Oncol. 2013; 6(1):17-24. doi:10.1593/tlo.12412
8. Sannachi L, Gangeh M, Tadayyon H, et al. Breast Cancer Treatment Response Monitoring Using Quantitative Ultrasound and Texture Analysis: Comparative Analysis of Analytical Models. Transl Oncol. 2019; 12(10):1271-1281. doi:10.1016/j.tranon.2019.06.004
9. Pfob A, Sidey-Gibbons C, Lee H B, et al. Identification of breast cancer patients with pathologic complete response in the breast after neoadjuvant systemic treatment by an intelligent vacuum-assisted biopsy. Eur J Cancer. 2021; 143:134-146. doi:10.1016/j.ejca.2020.11.006
10. Rueth N M, Lin H Y, Bedrosian I, et al. Underuse of Trimodality Treatment Affects Survival for Patients With Inflammatory Breast Cancer: An Analysis of Treatment and Survival Trends From the National Cancer Database. Journal of Clinical Oncology. 2014; 32(19):2018-2024. doi:10.1200/JCO.2014.55.1978
11. Scholl S M, Fourquet A, Asselain B, et al. Neoadjuvant versus adjuvant chemotherapy in premenopausal patients with tumours considered too large for breast conserving surgery: Preliminary results of a randomised trial: S6. Eur J Cancer. 1994; 30(5):645-652. doi:10.1016/0959-8049(94) 90537-1
12. Meng X, Chang X, Wang X, Guo Y. Development and Validation a Survival Prediction Model and a Risk Stratification for Elderly Locally Advanced Breast Cancer. Clin Breast Cancer. Published online 2022. doi:10.1016/j.clbc.2022.06.002
13. Sethi D, Sen R, Parshad S, Sen J, Khetarpal S, Garg M. Histopathologic changes following neoadjuvant chemotherapy in locally advanced breast cancer. Indian J Cancer. 2013; 50(1):58. doi:10.4103/0019-509X.112301
14. Chuthapisith S, Eremin J M, El-Sheemy M, Eremin O. Neoadjuvant chemotherapy in women with large and locally advanced breast cancer: Chemoresistance and prediction of response to drug therapy. The Surgeon. 2006; 4(4):211-219. doi:10.1016/S1479-666X(06)80062-4
15. Sethi D, Sen R, Parshad S, Sen J, Khetarpal S, Garg M. Histopathologic changes following neoadjuvant chemotherapy in locally advanced breast cancer. Indian J Cancer. 2013; 50(1):58. doi:10.4103/0019-509X.112301
16. Haque W, Verma V, Hatch S, Suzanne Klimberg V, Brian Butler E, Teh B S. Response rates and pathologic complete response by breast cancer molecular subtype following neoadjuvant chemotherapy. Breast Cancer Res Treat. 2018; 170(3):559-567. doi:10.1007/s10549-018-4801-3
17. Tudorica A, Oh K Y, Chui S Y C, et al. Early Prediction and Evaluation of Breast Cancer Response to Neoadjuvant Chemotherapy Using Quantitative DCE-MRI. Transl Oncol. 2016; 9(1):8-17. doi:10.1016/j.tranon.2015.11.016
18. Tahmassebi A, Wengert G J, Helbich T H, et al. Impact of Machine Learning With Multiparametric Magnetic Resonance Imaging of the Breast for Early Prediction of Response to Neoadjuvant Chemotherapy and Survival Outcomes in Breast Cancer Patients. Invest Radiol. 2019; 54(2):110-117. doi:10.1097/RLI.0000000000000518
19. Moghadas-Dastjerdi H, Sha-E-Tallat H R, Sannachi L, Sadeghi-Naini A, Czarnota G J. A priori prediction of tumour response to neoadjuvant chemotherapy in breast cancer patients using quantitative C T and machine learning. Sci Rep. 2020; 10(10936). doi:10.1038/s41598-020-67823-8
20. Taleghamar H, Jalalifar S A, Czarnota G J, Sadeghi-Naini A. Deep learning of quantitative ultrasound multi-parametric images at pre-treatment to predict breast cancer response to chemotherapy. Sci Rep. 2022; 12(1):2244. doi:10.1038/s41598-022-06100-2
21. Tran W T, Gangeh M J, Sannachi L, et al. Predicting breast cancer response to neoadjuvant chemotherapy using pretreatment diffuse optical spectroscopic texture analysis. Br J Cancer. 2017; 116(10):1329-1339. doi:10.1038/bjc.2017.97
22. Taleghamar H, Moghadas-Dastjerdi H, Czarnota G J, Sadeghi-Naini A. Characterizing intra-tumor regions on quantitative ultrasound parametric images to predict breast cancer response to chemotherapy at pre-treatment. Sci Rep. 2021; 11(1):14865. doi:10.1038/s41598-021-94004-y
23. Hayashi M, Yamamoto Y, Iwase H. Clinical imaging for the prediction of neoadjuvant chemotherapy response in breast cancer. Chin Clin Oncol. 2020; 9(3):31. doi:10.21037/cco-20-15
24. Romeo V, Accardo G, Perillo T, et al. Assessment and Prediction of Response to Neoadjuvant Chemotherapy in Breast Cancer: A Comparison of Imaging Modalities and Future Perspectives. Cancers (Basel). 2021; 13(14). doi:10.3390/cancers13143521
25. Saednia K, Lagree A, Alera M A, et al. Quantitative digital histopathology and machine learning to predict pathological complete response to chemotherapy in breast cancer patients using pre-treatment tumor biopsies. Sci Rep. 2022; 12(1):9690. doi:10.1038/s41598-022-13917-4
26. Rehman A, Ahmed Butt M, Zaman M. A Survey of Medical Image Analysis Using Deep Learning Approaches. In: 2021 5th International Conference on Computing Methodologies and Communication (ICCMC). IEEE; 2021:1334-1342. doi:10.1109/ICCMC51019.2021.9418385
27. Kim K G. Book Review: Deep Learning. In: Healthcare Informatics Research. Vol 22.; 2016:351-354. doi:10.4258/hir.2016.22.4.351
28. Zadeh Shirazi A, Fornaciari E, Bagherian N S, Ebert L M, Koszyca B, Gomez G A. DeepSurvNet: deep survival convolutional network for brain cancer survival rate classification based on histopathological images. Med Biol Eng Comput. 2020; 58(5):1031-1045. doi:10.1007/s11517-020-02147-3
29. Saednia K, Tran W T, Sadeghi-Naini A. Automatic characterization of breast lesions using multi-scale attention-guided deep learning of digital histology images. Comput Methods Biomech Biomed Eng Imaging Vis. Published online 2022:1-9. doi:10.1080/21681163.2022.2058415
30. Coudray N, Ocampo P S, Sakellaropoulos T, et al. Classification and mutation prediction from non-small cell lung cancer histopathology images using deep learning. Nat Med. 2018; 24(10):1559-1567. doi:10.1038/s41591-018-0177-5
31. Echle A, Rindtorff N T, Brinker T J, Luedde T, Pearson A T, Kather J N. Deep learning in cancer pathology: a new generation of clinical biomarkers. Br J Cancer. 2021; 124(4):686696. doi:10.1038/s41416-020-01122-x
32. Dimitriou N, Arandjelović O, Caie P D. Deep Learning for Whole Slide Image Analysis: An Overview. Front Med (Lausanne). 2019; 6(264). doi:10.3389/fmed.2019.00264
33. Clunie D, Hosseinzadeh D, Wintell M, et al. Digital Imaging and Communications in Medicine Whole Slide Imaging Connectathon at Digital Pathology Association Pathology Visions 2017. J Pathol Inform. 2018; 9(1):6. doi:10.4103/jpi.jpi_1_18
34. Chollet F. Xception: Deep Learning With Depthwise Separable Convolutions. In: Proceedings of the IEEE Conference on Computer Vision and Pattern Recognition (CVPR); 2017.
35. Szegedy C, Vanhoucke V, Ioffe S, Shlens J, Wojna Z. Rethinking the Inception Architecture for Computer Vision. In: Proceedings of the IEEE Conference on Computer Vision and Pattern Recognition (CVPR).; 2016.
36. Deng J, Dong W, Socher R, Li L J, Li K, Fei-Fei L. Imagenet: A large-scale hierarchical image database. In: 2009 IEEE Conference on Computer Vision and Pattern Recognition; 2009:248-255.
37. Vaswani A, Shazeer N, Parmar N, et al. Attention is All you Need. In: Guyon I, Luxburg Uvon, Bengio S, et al., eds. Advances in Neural Information Processing Systems. Vol 30.Curran Associates, Inc.; 2017. https://proceedings.neurips.cc/paper/2017/file/3f5ee243547dee91fbd053c1c4a845aa-Paper.pdf
38. Dzmitry Bahdanau, Kyunghyun Cho, Yoshua Bengio. Neural Machine Translation by Jointly Learning to Align and Translate. In: Third International Conference on Learning Representations (ICLR2015).; 2015.
39. Dosovitskiy A, Beyer L, Kolesnikov A, et al. An Image is Worth 16×16 Words: Transformers for Image Recognition at Scale. Published online 2020. http://arxiv.org/abs/2010.11929
40. Khan S, Naseer M, Hayat M, Zamir S W, Khan F S, Shah M. Transformers in Vision: A Survey. ACM Comput Surv. 2022; 54(10s):1-41. doi:10.1145/3505244
41. Paul S, Chen P Y. Vision Transformers Are Robust Learners. Proceedings of the AAAI Conference on Artificial Intelligence. 2022; 36(2):2071-2081. doi:10.1609/aaai.v3612.20103
42. Woo S, Park J, Lee J Y, Kweon I S. CBAM: Convolutional Block Attention Module. Proceedings of the European Conference on Computer Vision (ECCV). Published online 2018:3-19.
43. Dai Z, Liu H, Le Q v, Tan M. CoAtNet: Marrying Convolution and Attention for All Data Sizes. In: Ranzato M, Beygelzimer A, Dauphin Y, Liang P S, Vaughan J W, eds. Advances in Neural Information Processing Systems. Vol 34. Curran Associates, Inc.; 2021:3965-3977. https://proceedings.neurips.cc/paper/2021/file/20568692db622456cc42a2e853ca21f8-Paper.pdf
44. Symmans W F, Peintinger F, Hatzis C, et al. Measurement of Residual Breast Cancer Burden to Predict Survival After Neoadjuvant Chemotherapy. Journal of Clinical Oncology. 2007; 25(28):4414-4422. doi:10.1200/JCO.2007.10.6823
45. Martel A L, Hosseinzadeh D, Senaras C, et al. An Image Analysis Resource for Cancer Research: PIIP-Pathology Image Informatics Platform for Visualization, Analysis, and Management. Cancer Res. 2017; 77(21). doi:10.1158/0008-5472.CAN-17-0323
46. Selvaraju R R, Cogswell M, Das A, Vedantam R, Parikh D, Batra D. Grad-CAM: Visual Explanations from Deep Networks via Gradient-Based Localization. In: 2017 IEEE International Conference on Computer Vision (ICCV). IEEE; 2017:618-626. doi:10.1109/ICCV.2017.74
47. Heindl A, Nawaz S, Yuan Y. Mapping spatial heterogeneity in the tumor microenvironment: a new era for digital pathology. Laboratory Investigation. 2015; 95(4):377-384. doi:10.1038/labinvest.2014.155
48. Ha S, Park S, Bang J I, Kim E K, Lee H Y. Metabolic Radiomics for Pretreatment 18F-FDG PET/CT to Characterize Locally Advanced Breast Cancer: Histopathologic Characteristics, Response to Neoadjuvant Chemotherapy, and Prognosis. Sci Rep. 2017; 7(1):1556. doi:10.1038/s41598-017-01524-7
49. Kolios C, Sannachi L, Dasgupta A, et al. MRI texture features from tumor core and margin in the prediction of response to neoadjuvant chemotherapy in patients with locally advanced breast cancer. Oncotarget. 2021; 12(14):1354-1365. doi:10.18632/oncotarget.28002
50. Duanmu H, Bhattarai S, Li H, et al. A spatial attention guided deep learning system for prediction of pathological complete response using breast cancer histopathology images. Bioinformatics. 2022; 38(19):4605-4612. doi:10.1093/bioinformatics/btac558
51. Lagree A, Shiner A, Alera M A, et al. Assessment of Digital Pathology Imaging Biomarkers Associated with Breast Cancer Histologic Grade. Curr Oncol. 2021; 28(6):4298-4316. doi:10.3390/curronco128060366
52. Bulten W, Kartasalo K, Chen P H C, et al. Artificial intelligence for diagnosis and Gleason grading of prostate cancer: the PANDA challenge. Nat Med. 2022; 28(1):154-163. doi:10.1038/s41591-021-01620-2
53. Taylor-Weiner A, Pokkalla H, Han L, et al. A Machine Learning Approach Enables Quantitative Measurement of Liver Histology and Disease Monitoring in NASH. Hepatology. 2021; 74(1):133-147. doi:10.1002/hep.31750
54. Shim W S, Yim K, Kim T J, et al. DeepRePath: Identifying the Prognostic Features of Early-Stage Lung Adenocarcinoma Using Multi-Scale Pathology Images and Deep Convolutional Neural Networks. Cancers (Basel). 2021; 13(13). doi:10.3390/cancers13133308

1. A system for automatically predicting chemotherapy response in a patient with cancer using a trained machine learning model, the system including:
a digital pathology imaging system for acquiring at least one pathology image;
a patch-level processing module for capturing local correlations within a tumor region in at least one pathology image patch, wherein the at least one pathology image patch is derived from at least one pathology image of a pre-treatment biopsy specimen, and extracting feature maps carrying relevant information of the at least one pathology image patch;
a tumor-level processing module for aggregating the local information for each tumor region;
a patient-level prediction module for applying the sequence of the local information for the tumor regions to derive global relations and predict the patient's response to chemotherapy.

2. The system of claim 1, wherein the patch-level processing module comprises a modified CoAtNet model with two convolutional components followed by two self-attention modules.

3. The system of claim 1, wherein a first sequence of feature maps for each tumor bed is generated by stacking the sorted patch-level feature maps of the corresponding tumor bed annotation in the associated at least one image.

4. The system of claim 3, wherein feature map sorting and sequence length thresholding strategies are applied, and the generated first feature map sequence is fed as input to the tumor-level processing module.

5. The system of claim 4, wherein the tumor-level processing module comprises a first prediction vision transformer (ViT) network for determining the relations in aggregated features of each tumor bed.

6. The system of claim 5, wherein the patient-level prediction module aggregates the information obtained at the tumor-level module.

7. The system of claim 6, wherein feature map sorting and sequence length thresholding strategies is applied to generate a second feature map sequence for input into a second prediction ViT network associated with the patient-level prediction module.

8. The system of claim 7, wherein the cancer is at least one of a breast cancer, liver cancer, prostate cancer and lung cancer.

9. A computerized method for automatically predicting chemotherapy response in a patient with cancer using a trained machine learning model, the method comprising:
with an imaging system, acquiring at least one image of a pre-treatment biopsy specimen,
identifying at least one region of interest (ROI) comprising a tumor within the at least one image;
applying the at least one image to a trained machine learning model to perform the steps of:
(a) capturing local correlations within each ROI and extracting feature maps with relevant information of pathology image patches;
(b) aggregating local information for each ROI; and
(c) using a sequence of the aggregate information for a plurality of ROIs to derive global relations and predict the patient's response to chemotherapy.

10. A computerized method for training processing circuitry using machine learning to predict chemotherapy response in a patient with cancer, the method comprising:
training a machine learning model to at least:
capture local correlations within at least one region of interest (ROI) comprising a tumor within at least one image acquired by an imaging system, and extract feature maps with relevant information of pathology image patches derived from the at least one image;
aggregate local information for each ROI; and
and predict the patient's response to chemotherapy by using a sequence of the aggregate information for a plurality of ROIs to derive global relations.

11. A non-transitory computer readable medium storing machine-interpretable instruction sets which when executed by a processor, cause the processor to perform a method for predicting chemotherapy response in a patient with cancer, the method comprising:
instantiating a machine learning model architecture;
receiving at least one pathology image patch derived from at least one image of a pre-treatment biopsy specimen acquired by an imaging system;
training the machine learning model architecture using the set of the at least one pathology image patches, the machine learning model architecture including:
a convolutional network architecture configured to capture local correlations within at least one region of interest (ROI) comprising a tumor within the at least one pathology image patch and extract feature maps with relevant information of pathology image patches;
a first encoder configured to aggregate local information for each ROI; and
a second encoder configured to use a sequence of the aggregate information for a plurality of ROIs to derive global relations and predict the patient's response to chemotherapy.
